(19) United States
(12) Patent Application Publication
(10) Pub. No.: US 2004/0115803 A1
(43) Pub. Date: Jun. 17, 2004

(54) Transgenic cells and animals for the study of the polarization of the immune response

(76) Inventors: Kader Thiam, Lyon (FR); Alexandre Fraichard, Lyon (FR); Christine Lapize-Gauthey, Vienne (FR)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/471,465

(22) PCT Filed: Mar. 8, 2002
(86) PCT No.: PCT/FR02/00837

(30) Foreign Application Priority Data
Mar. 9, 2001 (FR) 01/03272

(51) Int. Cl. C12N 5/06

(52) U.S. Cl. 435/354

(57) ABSTRACT

The invention relates to a transgenic non-human animal cell expressing at least one transgene coding for at least one reporter protein, characterized in that the expression of said reporter protein is correlated to the expression of at least one protein which is naturally produced by said cell and which is specific for a type of polarization of the immune response and/or an effector function of the immune response. The invention also relates to a corresponding transgenic animal. According to the invention, the cell and the transgenic animal can be used in a method for characterizing the type of immune response, i.e. Th1 and Th2, caused by an immunogene, a pathogen or chemical agent.

This invention relates to the domain of biology and more particularly to animal transgenesis. The invention is related to a non-human transgenic animal cell expressing at least one coding transgene for at least one reporter protein, characterized in that the expression of the said reporter protein is correlated to the expression of at least one protein produced naturally by the said cell and specific to a polarisation type of the immune response and/or an effector function of the immune response. The invention also relates to the corresponding transgenic animal. The cell and the transgenic animal according to the invention can be used in a process to characterize the immune response type, particularly Th1 and Th2, induced by an immunogen. A method of screening compounds that modulate the polarisation type of the immune response and/or the type of effector function of the immune response is also claimed.

A specific immune response against pathogens is the result of a combination of multiple parameters that include precocious production of some cytokins and chemokins, induction of synthesis of surface cellular molecules, cell/cell interactions and activation of a number of different cell types. Typically, a distinction should be made between two main types of acquired immunity depending on the nature of the pathogen. Intracell microorganisms such as viruses, some types of bacteria and protozoans induce immune responses mediated by T cytotoxic cells and production of inflammatory cytokins such as interferon γ, interleukin 1, TNF α, the purpose of which is to induce resistance to infection and/or to eliminate infected cells. Extra-cellular pathogens such as helminths induce a humoral response associated with the production of specific antibodies that neutralize the antigen.

Appropriate polarisation of either of the responses is crucial to achieve efficient elimination of the pathogen. This polarisation requires differentiation of naive CD4+ T cells into Th1/Th2 effector cells. This step is a major element in the decision about the type of immune response (Mossmann et al., 1986; Mossmann et al., 1989; Romagnani, 1999). The aptitude of the different subtypes of T cells to direct immune effector responses is due to an exclusive combination of cytokins expressed partly in a subtype of particular Th cells. Thus, in man and also in the mouse, Th1 cells preferably produce interleukin 2 and interferon γ; these Th1 cells cause a retarded hypersensitivity type response, while Th2 cells secrete interleukin 4, interleukin 5, interleukin 10 and induce activation of B cells and the production of antibodies. An unbalance in Th1/Th2 responses was observed in a variety of clinical situations, for example such as atopic allergy (mediated by IgE), and particularly asthma, allergic rhinitis, and also graft rejects, diseases of the graft against the host, leishmanioses, leprosy, tuberculosis and chronic inflammatory diseases such as Crohn's disease, reactive arthritis, insulino-dependent diabetes. It has been suggested that the Th1/Th2 response can be manipulated to develop better quality vaccines, new anti-tumoral approaches and alternative immunoprophylactic strategies for therapy of allergies and auto-immune diseases. Thus, a constant effort is made to increase the understanding of mechanisms involving the Th1/Th2 ratio or to discover new molecules that could modify these two processes. Th1/Th2 responses are typically studied by checking the cell type and the expression of cell surface markers, and the production of cytokins either in the serum, or in lymphoid systems. Methods such as the ELISA method, quantitative RT-PCR methods and flux cytometry are commonly used. Most of these tests are long and laborious since they require a large number of manipulations. These methods are not suitable for testing a large number of experimental conditions; these methods can also disturb the analysis since they can modify the natural response of cells, for example as in the case of methods that imply ex vivo reactivation of T cells. To overcome these various disadvantages according to prior art, animal models enabling fast characterisation of the type of immune response would be extremely valuable tools for carrying out these studies. This type of animal model provides a means of obtaining a fast, easy, reproducible and safe response from the natural biological response and would make it possible to reliably identify Th1/Th2 parameters.

This is why the inventors propose to develop new models by manipulating the animal genome, and particularly murine, so that the immune response type will be represented directly by the expression of a specific reporter gene. Therefore, this invention is intended to supply a non-human transgenic animal cell expressing at least one coding transgene for at least one reporter protein, characterized in that the expression of the said reporter protein is correlated to the expression of at least one protein produced naturally by the said cell and specific to a type of polarisation of the immune response and/or an effector function of the immune response.

The phrase “expression of the said reporter protein is correlated to the expression of at least one naturally produced protein” means that the expression of the reporter protein is directly or indirectly associated with the expression of the naturally produced protein. Thus, when the naturally produced protein is expressed, the reporter protein is also expressed, and when the naturally produced protein is not expressed, the reporter protein is not expressed. Preferably, the expression ratio of the reporter protein will be directly proportional to the expression ratio of the naturally produced protein. Thus for example, when the expression ratio of the naturally produced protein is high, the expression ratio of the reporter protein will be high, and when the expression ratio of the naturally produced protein is low the expression ratio of the reporter protein will be low. As will be seen later, this correlation is preferably obtained by putting the reporter gene under the control of elements regulating expression of the coding gene for the naturally produced protein, without inhibiting the expression of this gene; in this case, the expression of the two genes is CIS regulated, in other words the expression of the reporter protein is directly associated with the expression of the naturally produced protein. This correlation may also be obtained by putting the reporter gene under the control of transcription regulation elements that can be induced by the naturally produced protein or one of the metabolic cascade proteins induced by the expression of the naturally produced protein. In this case, the expression of the two genes is TRANS regulated, in other words the expression of the reporter protein is indirectly associated with the expression of the naturally produced protein.

The cell according to this invention is also characterized by the fact that for each type of polarisation of the immune type and/or for each type of effector function, there is a separate reporter protein that makes it easy and fast to simultaneously distinguish the polarisation type(s) of the immune response and/or the type of effector functions involved in the response of the cell or the animal according to the invention to one or several immmunogenic or pathogenic agents.

Polarisation of the immune response denotes the nature of the immune response (cellular or humoral) following the first encounter with an antigen. Among immune responses to cellular mediation, a distinction should be made between different polarisation subtypes such as the immune response mediated by auxiliary T lymphocytes (“T helper”), T repressor lymphocytes (Groux et al., 1997), T cytotoxic lymphocytes, NK cells, K cells and the humoral type, can be distinguished (this list is not exhaustive). Preferably, a “T helper” polarisation type will be chosen from among types Th0 (Firestein et al., 1989), Th1, Th2, Th3, Tr1. Preferably, it will be a type Th1 (Mossmann et al., 1986; Mossmann et al., 1989; Del Prete et al., 1991; Wiernenga et al., 1990; Yamamura et al., 1991; Robinson et al., 1993) and Th2. Among the humoral immune response, note the immune response mediated by B lymphocytes.

Types of effector function of the immune response according to the meaning of this invention include CTL activities or functions, phagocyte activities or functions, cytotoxic activities or functions, immunosuppressive activities or functions, antigen presentation activities or functions, cellular activation activities or functions; this list is not exhaustive.

An effector function denotes a cellular function of one or several cell types with an effector result on a given type of target cell. These functions participate in setting a protective immunity and/or development of an appropriate immune response, for example such as the control of an exacerbated immune response.

A “naturally expressed protein” denotes a protein expressed from a gene present in its natural chromatinian environment, which consequently includes a natural regulation of its expression. Thus, preferably, the coding gene for the naturally expressed protein was not introduced into the cell by transfection or transgenesis; it is a so-called endogenic gene.

The invention can be made in any mammal cell competent for homologous recombination. Preferably, rodent cells will be used, particularly mouse, rat, hamster and guinea pig cells. Preferably, mouse cells will be used. Alternately, primate cells (not taken from man) will be used, for example taken from monkeys, chimpanzees, macaques, baboons. The cells used may also be bovine, caprinae, ovine, porcine cells, and particularly small pigs, horses and rabbits.

Cells according to the invention may be functionally defined as being capable of making a homologous recombination of the exogenic DNA fragment(s) that contain at least one and preferably two regions(s) with sequence homologies with an endogenic cellular DNA sequence. This type of cells naturally contains endogenic recombinases or cells that have been genetically modified to contain them or to contain the necessary compounds to recombine the DNA.

Preferably, among the various cells according to the invention, it is worth mentioning all cellular types naturally expressing specific proteins involved in one or several types of polarisation of the immune response and/or specific proteins involved in cellular communication mechanisms and in setting of one or several effector functions of the immune response. Coding genes for its specific proteins, called endogenic genes in this invention, preferably code intracellular or membrane proteins involved in intracellular signalling and in cellular communication, for soluble proteins. These are immune system proteins, for example such as cytokins, chemokins, lymphokins, cellular surface proteins (CD differentiation markers, membrane receivers), proteins involved in the transduction of the signal associated with different receiver types, and/or in activation or target genes. According to one particular preferred embodiment of the invention, the naturally produced proteins specific to Th1 type polarisation is chosen non-exhaustively from among interleukin 2 (IL-2), interleukin 12 (IL-12), interleukin 18 (IL-18), interferon γ, TNF-β, TNF-αT-bet, STAT-4, the β chain of the interferon γ receiver (IFN-γ), IL-12 and IL-18 receiver chains, RANTES, MIP-1α, MIP-1β, CD26, the β2 chain of the interleukin 12 receiver (IL-12Rβ₂), CCR5, CCR2, CXCR3. Preferably, the specific protein for type Th1 polarisation is IFN-γ. The specific protein for type Th2 polarisation is preferably chosen from the group composed non-exhaustively of interleukin 3 (IL-3), interleukin 4 (IL-4), interleukin 5 (IL-5), interleukin 10 (IL-10), interleukin 13 (IL-13), GATA-3, STAT-6, c-maf, NFAT, NIP45, CD30, CD26L, ST2L, CCR3, CCR4, CCR8, CXCR4, CRTH2, STIF (WO 98 46 638). Preferably, the specific protein for type Th2 polarisation is interleukin 4 (IL-4). Among these cells, it is worth noting cells in the immune system and non-exhaustively T lymphocytes, NK cells, K cells, B lymphocytes, basophiles, mastocytes, macrophages, eosinophiles, monocytes, neutrophiles, blood platelets, Langerhans cells, dendritic cell monocytes. For example, cells according to the invention may also be neuron cells. We should also mention cells that under some culture conditions, or after differentiation or genetic manipulation, are capable of expressing specific proteins involved in one or several types of polarisation of the immune response and/or specific proteins involved in one or several effector functions of the immune response. We could mention haematopoietic stem cells, totipotential embryonic stem cells (ES cells) or pluripotential cells. These stem cells may be differentiated as cells expressing specific proteins according to the invention. A stem cell means any type of undifferentiated multipotential or pluripotential cell that can be cultivated in vitro in a prolonged manner without losing its characteristics, and that can be differentiated into one or several cell types when placed under defined culture conditions. Thus, when the cell according to the invention is an ES cell or haematopoietic cell, it will be possible to induce differentiation of this cell into different cell types that could express the specific protein(s) of the immune response, for example such as neuron cells and immune system cells, and more precisely mastocytes, basophiles, monocytes, eosinophiles, T lymphocytes, NK cells, K cells, B lymphocytes, Langerhans cells, blood platelets, monocytes of dendritic cells.

When embryonic stem cells (ES) have to be used, for example for production of the transgenic animal according to the invention, a cell line of ES cells may be used or fresh embryo cells may be obtained from a host animal according to the invention, usually a mouse, rat, hamster or guinea pig. This type of cell is cultivated on a layer of appropriate feeder fibroblasts or on gelatine, in the presence of appropriate growth factors such as a Leukaemia Inhibiting Factor (LIF).

More generally, cells according to the invention correspond to all animal cells (preferably mammals) except for human cells. Therefore examples of mammal cells competent for recombination include fibroblasts, endothelial cells, epithelial cells, cells normally cultivated in the laboratory such as Hela cells, CHO (Chinese Hamster Ovary) cells, Dorris, AE7, D10.64, DAX, D1.1, CDC25 for example.

For the purposes of the description of this invention, transgenic denotes a cell comprising a transgene. “Transgene” or exogenic nucleic acids sequence or exogenic gene denotes genetic material that has been or will be artificially inserted into the genome of a mammal, particularly into a mammal cell cultivated in vitro or into a living mammal cell, or that will be maintained in the said cell in episomal form. Preferably, the transgene according to this invention comprises at least one sequence that could be transcribed or transcribed and translated into protein. The transgene(s) according to the invention or their expression does (do) not affect operation of the biological network of the immune system, nor more generally operation of the biological network of the cell. The transgene may be cloned in a cloning vector that enables its propagation into a host cell, and/or optionally in an expression vector to enable expression of the transgene. Recombining DNA technologies used for construction of the cloning vector and/or the expression vector according to the invention are the same technologies that are known and commonly used by those skilled in the art. Standard techniques are used for cloning, isolation of DNA, amplification and purification; enzyme reactions involving ligase DNA, polymerase DNA and restriction endonucleases are carried out according to the manufacturer's recommendations. These and other techniques are generally made according to Sambrook et al., 1989). The vectors include plasmides, cosmides, phagemides, bacteriophages, retroviruses and other animal viruses, artificial chromosomes such as YAC, BAC, HAC and other analogue vectors.

Methods of generating transgenic cells according to the invention are well known to those skilled in the art (Gordon et al., 1989). Various techniques for transfecting mammal cells have been described (see review by Keon et al., 1990). The transgene according to the invention, optionally included in a linearized or non-linearized vector or in the form of a vector fragment, may be introduced into the host cell by standard methods for example such as micro-injection into the nucleus (U.S. Pat. No. 4,873,191), transfection by precipitation with calcium phosphate, lipofection, electroporation (Lo, 1983), thermal shock, transformation with cationic polymers (PEG, polybrene, DEAE-Dextran, etc.), viral infection (Van der Putten et al., 1985), sperm (Lavitrano et al., 1989).

According to one preferred embodiment of the invention, the transgenic cell according to the invention is obtained by gene targeting of the transgene(s) at one or more of the genome sequences of the host cell. More precisely, the genome is inserted in a stable manner by homologous recombination at homologous sequences in the genome of the host cell. When the objective is to obtain a transgenic cell in order to produce a transgenic animal, the host cell is preferably an embryonic stem cell (ES cell) (Thompson et al., 1989).

Gene targeting represents the directed modification of a chromosome locus by homologous recombination with an exogenic DNA sequence homologous with the targeted endogenic sequence. A distinction is made between different types of gene targeting. Thus, gene targeting may be used to modify, and usually increase, the expression of one or several endogenic genes, or to replace an endogenic gene by an exogenic gene, or to place an exogenic gene under the control of elements regulating the gene expression of the particular endogenic gene that remains active. In this case, gene targeting is called “Knock-in” (KI). Alternatively, gene targeting may be used to reduce or eliminate the expression of one or several genes, and this type of gene targeting is called “Knock-out” (KO) (see Bolkey et al., 1989).

According to this invention, integration of the said cell of the said coding transgene for at least one reporter protein into the genome forms a “Knock-in”; it is done at the said coding endogenic gene(s) for one or more proteins specific to a type of polarisation of the immune response and/or a type of effector function of the immune response without the said transgene inhibiting the expression of the said endogenic gene, and without the expression of the said transgene affecting the biological network of the cell. The cell according to the invention is characterized in that the transgene is integrated in a stable manner into the genome of the said cell, and that its expression is controlled by regulation elements of the coding endogenic gene for the said naturally produced protein specific to a type of polarisation of the immune response and/or an effector function of the immune response. Stable integration means insertion of the transgene in the genomic DNA of the cell according to the invention. The transgene thus inserted is then transmitted to the cell descendance. The transgene is integrated on the upstream side, or on the downstream side, or in the middle of the target endogenic gene. Preferably, the transgene comprises a sequence to retrieve the translation, for example such as an IRES (internal ribosome entry site) sequence (Mountford et al., 1995; Zhu et al., 1999; Liu et al, 2000), the said sequence being located between the coding sequence of the said reporter protein and the coding sequence of the said protein specific to a type of polarisation of the immune response and/or an effector function of the immune response. According to one preferred embodiment, the cell according to the invention expresses one or several transgenes, and preferably two coding transgenes each coding for a distinct reporter protein, the expression of each reporter protein being specific to a type of polarisation of the immune response and preferably of the Th1 or Th2 type. More particularly, the non-human transgenic animal cell according to the invention is characterized in that it expresses (a) a first coding transgene for a first reporter protein, the said first transgene being integrated by homologous recombination (“Knock-in”) in a coding endogenic animal gene for a specific protein with type Th1 polarisation of the immune response without inhibiting the expression of the said endogenic gene, the expression of the said first transgene being correlated with the expression of the said endogenic animal gene; and/or (b) a second coding transgene for a second reporter protein, distinct from the said first reporter protein, the said second transgene being integrated by homologous recombination (“Knock-In”) at a coding endogenic gene for a specific protein with type TH2 polarisation of the immune response, without inhibiting the expression of the said endogenic gene, the expression of the said second transgene being correlated with the expression of the said endogenic gene.

To achieve homologous recombination, it is necessary for the transgene to contain at least one DNA sequence comprising at least the reporter gene, possibly with the required genetic modifications and optionally with one or several positive or negative selection genes, and also DNA regions (preferably two) homologous with the target locus, located on each side of the portion of the reporter gene. “Homologous or substantially homologous DNA regions” denotes two DNA sequences that, after optimal alignment and after comparison, are identical for at least about 75% of nucleotides, at least about 80% of nucleotides, normally at least about 90 to 95% of nucleotides, and even better at least about 98 to 99.5% of nucleotides. For the purposes of this invention, “identity percentage” between two nucleic acid sequences means a percentage of identical nucleotides in the two sequences to be compared, obtained after the best alignment, this percentage being purely statistical and the differences between the two sequences being distributed at random and over their entire length. “Best alignment” or “optimal alignment” denotes the alignment for which the identity percentage determined as described below is highest. Sequences between two nucleic acid sequences are traditionally compared after optimally aligning these sequences, the said comparison being made by segment or by “comparison window” to identify and compare local regions with a similar sequence. The optimal alignment of sequences for the comparison can be made manually, or using Smith and Waterman's local homology algorithm (1981), or Neddleman and Wunsch's local homology algorithm (1970), or Pearson and Lipman's similarity search method (1988), or using computer software making use of these algorithms (GAP, BESTFIT, BLAST P, BLAST N, FASTA and TFASTA in the Wisconsin Genetics Software Package, Genetics Computer Group, 575 Science Dr., Madison, Wis.). The BLAST program will preferably be used with the BLOSUM 62 matrix to obtain optimum alignment. The PAM or PAM250 matrices could also be used. The identity percentage between two nucleic acid sequences is determined by comparing these two optimally aligned sequences, the sequence of nucleic acids or amino acids to be compared possibly including additions or deletions compared with the reference sequence for optimum alignment between these two sequences. The identity percentage is calculated by determining the number of identical positions for which the nucleotide or the amino acid residue is identical in the two sequences, by dividing this number of identical positions by the total number of compared positions, and then multiplying the result obtained by 100 to obtain the identity percentage between these two sequences. The phrase “nucleic sequences with an identity percentage of at least 85%, preferably at least 90%, 95%, 98% and 99% after optimal alignment with a reference sequence”, denotes nucleic sequences with some modifications from the reference nucleic sequence, particularly such as a deletion, a truncation, an elongation, a chimeric fusion, and/or a substitution, particularly one-off, and for which the identity percentage after optimal alignment with the reference nucleic sequence is at least 85%, and preferably at least 90%, 95%, 98% and 99%. The length of homology regions is partly dependent on the degree of homology. This is due to the fact that a reduction in the homology quantity causes a reduction in the frequency of homologous recombination. If there are any non-homologous regions in the portions of homologous sequences, it is preferable if this non-homology is restricted to discrete portions rather than extending over the entire portion of the homologous sequence. In all cases, the length of the homology region needs to increase as the degree of homology reduces, to facilitate homologous recombination. Although as little as 14 pb homology at 100% is sufficient for homologous recombination in bacteria, longer portions of homologous sequences are generally preferred in mammal cells. These portions are at least 250 pb, 500 pb, 750 pb, 1 000 pb, 1500 pb, 1750 pb, 2000 pb, 2500 pb, 3000 pb, 4000 pb and preferably 5000 pb for each portion of the homologous sequence. According to the invention, the DNA fragments may be any size. The minimum required size depends on the need for at least one sufficiently long homology region to facilitate homologous recombination. The size of DNA fragments is at least about 2 kb, and preferably at least about 3 kb, 5 kb, 6 kb.

The transgene is not limited to one particular sequence of DNA. Thus the homologous DNA sequences present in the transgene can be of a purely synthetic origin (for example produced by a method using DNA synthesizer), or they can derive from mRNA sequences by reverse transcription, or can derive directly from genomic DNA. When the homologous DNA is derived from RNA sequences by reverse transcription, it may or may not contain all or a part of the non-coding sequences such as introns, depending on whether the corresponding RNA molecule has or has not undergone, partial or complete splicing. Preferably, the homologous DNA sequences used for producing the homologous recombination comprise genomic DNA sequences rather than cDNA. In fact, the important cis-regulatory sequences present in the introns, the distal regions, the promoter regions can be present. The sequences deriving from genomic DNA generally code for at least one portion of the gene but can alternatively code for non-transcribed regions or region of a non-rearranged genetic locus such as the immunoglobin loci or the T-cell receptor loci. Generally, the genomic DNA sequences include a sequence coding for an RNA transcript. Preferably, the RNA transcript codes for a polypeptide; preferably, it is an γ-interferon and interleukin IL-4. According to one preferred embodiment, the transgene comprises all or part of the murine IL-4 gene characterized in that in the non-coding 3′ end of the murine IL-4 gene, an IRES sequence, a sequence coding for an auto-fluorescence protein, plus or minus a positive selection box framed by sites-specific to the action of the recombinases, for example a Lox/neo-TK/Lox or lox/Neo/10× or FRT/Neo-TK/FRT or FRT/Neo/FRT can also be present at the 5′ position of the reporter gene, can be inserted sequentially and characterized in that a negative selection box containing, for example, the DTA and/or TK gene or genes is present at at least one of the transgene ends. When the specific protein of the Th2 protein polarization is IL-4, then the auto-fluorescence protein is preferably RFP. According to another embodiment, the transgene comprises all or part of the murine γ-IFN gene, characterized in that at the non-coding 3′ end of the murine γ-IFN gene an IRES sequence, a sequence coding for an auto-fluorescent protein, a positive selection box whether framed or not with sites specific for recombinase action, for example, a Lox/Neo-TK/Los or lox/Neo/lox or FRT/Neo-TK/FRT or FRT/Neo/FRT box can also be present at the 5′ position of the reporter gene, can be inserted sequentially and characterized in that a negative selection box containing, for example, the DTA and/or TK gene or genes is present at at least one of the transgene ends. When the protein specific to the Th1 polarization is IFN-γ, then the auto-fluorescent protein is preferably GFP.

The transgene can also be as small as several hundreds of cDNA base pairs or as large as several hundred thousands of base pairs of gene locus comprising the coding exon-intron sequence and the regulation sequences necessary for obtaining spatially and/or temporally controlled expression. Preferably, the recombinant DNA segment is of a size ranging from 2.5 kb and 1,000 kb. Whatever it is, the recombinant DNA segments can be less than 2.5 kb and greater than 1,000 kb.

The transgene of the present invention is preferably in the native form; in other words, derived directly from an exogenous DNA sequence naturally present in an animal cell. This native DNA sequence can be modified, for example, by insertion of restriction sites necessary for cloning and/or by insertion of site-specific recombination sites (lox or flp sites). Alternatively, the transgene of the present invention can have been created artificially in vitro using recombinant DNA techniques, by combining, for example, portions of genomic DNA and cDNA. This is a chimeric transgene. The DNA sequence according to the invention, in native or chimeric form, can be mutated by using the techniques well known to the specialist in the field. These mutations can affect the amino acid sequences for the coding sequences.

When the cells have been transformed by the transgene, they can be cultured in vitro or even used for producing transgenic animals. After transformation, the cells are seeded on a culture medium and/or in a suitable medium. The cells containing the construction can be detected by using a selective medium. After a sufficient time allowing the colonies to establish, they are harvested and analyzed in order to determine if a homologous recombination event and/or integration of the construction has occurred. In order to perform the screening of clones having satisfied the homologous recombination, the positive and negative markers, called selection genes, can be inserted into the homologous recombination vector. Different systems of selection of cells having produced a homologous recombination event have been described; the first system described that uses positive/negative selection vectors can be cited (Mansour et al., 1988; Capecci, 1989).

Selection gene is understood to designate a gene that makes it possible for cells containing it to be specifically selected plus or minus the presence of a corresponding selective agent. To illustrate this, a resistance gene coding for antibiotic resistance can be used as a positive selection marker gene that allows a host cell to be positively selected in the presence of the corresponding antibiotic. A variety of positive and negative markers are known to the specialist in the field (for a review of these, please refer to the U.S. Pat. No. 5,627,059 patent). This selection gene can be located either on the inside or on the outside of the linearized transgene. When the selection gene is located within the transgene; in other words, between the 5′ and the 3′ ends of the transgene, it can be present in the form of a genic entity different from the reporter gene according to the invention. In this case, the selection gene is operationally linked with the DNA sequences making possible its expression; in the alternative, the selection gene can be placed under the control of sequences regulating expression of said reporter gene. These sequences, known to the specialist in the field, correspond particularly to the promoter sequences, optionally to the activator sequences and to transcription termination signals. Optionally, the selection gene can constitute a fusion gene with the reporter gene. Said fusion gene is thus operationally linked with the DNA sequences allowing control of expression of said fusion gene. According to another embodiment of the invention, the selection gene is located at the 5′ and 3′ ends of the transgene so that if a homologous recombination event occurs, the selection gene is not integrated into the cellular genomic DNA; in this case, the selection gene is a negative selection gene (for a review of this, see the U.S. Pat. No. 5,627,059 patent).

Said positive selection gene according to the invention is preferably chosen from among the antibiotic resistance genes. Of the antibiotics, neomycin, tetracycline, ampicillin, kanamycin, pleomycin, bleomycin, hygromycin, chloramphenicol, carbenicillin, geneticin, puromycin can be mentioned non-exhaustively. The resistance genes corresponding to these antibiotics are known to the specialist in the field; by way of example, the neomycin gene renders cells resistant to the presence of the antibiotic G418 in the culture medium. The positive selection gene can be similarly selected from the HisD gene, the corresponding selective agent being histidinol. The positive selection gene can also be selected from the guanine-phophoribosyl transferase (GpT) gene, whose corresponding selective agent being xanthine. The positive selection gene can also be selected from the hypoxanthine-phosphoribosyl transferase (HPRT) gene, whose corresponding selective agent being hypoxanthine.

Said negative selection gene according to the invention is preferably chosen from the 6-thioxyanthine or thymidine kinase (TK) gene (Mzoz et al., 1993), the genes coding for bacterial or viral toxins such as, for example, exotoxin A of Pseudomonas, diphtheria toxin (DTA), cholera toxin, anthrax toxin of the Bacillus, Pertussus toxin, Shiga toxin of Shigella, the toxin related to the Shiga toxin, the toxins of Escherichia coli, colicine A, d-endotoxins. Cytochrome p450 of the rate and cyclophosphamide (Wei et al., 1994), the purine nucleoside phosphorylase of Escherichia coli (E. coli), and 6-methcylpurine deoxyribonucleoside (Sorscher et al., 1994), the cytosine deaminases (Cdase) or uracil phosphoribosyl transferase (URPTase) can also be mentioned and can be used with 5-fluorocytosine (5-FC).

The selection marker or markers used to enable identification of the homologous recombination events can consequently affect genic expression, and can be eliminated, if required, by using site-specific recombinases such as Cre recombinase specific to Lox sites (Sauer, 1994; Rajewsky et al., 1996; Sauer, 1998) or FLP specific to the FRT sites (Kilby et al., 1993).

The positive colonies; in other words, those containing cells in which at least one homologous recombination event has occurred, are identified by Southern blot analysis and/or by PCR techniques. The rate of expression in isolated cells or transgenic cells according to the invention, the mRNA corresponding to the transgene can also be determined by techniques comprising Northern blot analysis, in situ hybridization analysis, RT-PCR. Similarly, the animal cells or tissues expressing the transgene can be identified using an antibody directed against the reporter protein. The positive cells can then be utilized for performing manipulations son the embryo and in particular by injection of the cells isolated by homologous recombination in the blastocysts. As relates to mice, the blastocysts are obtained from 4 to 6 week superovulated females. The cells are trypsinized and the modified cells are injected into the blastocell of a blastocyst. After injection, the blastocysts are introduced into the uterine horn of pseudogestating females. The females are allowed to reach term and the resulting litter is analyzed in order to determine the presence of mutant cells having the construct. Analysis the different genotype or phenotype between the newborn fetus and the cells of the blastocyst or the ES cells make it possible to detect the chimeric newborn. The chimeric fetuses are then reared to adulthood. The chimeras or chimeric animals are animals in which only one sub-population of cells has an altered genome. The chimeric animals have the modified gene or genes, are generally crossed with each other or with a wild-type animal in order to obtain heterozygous or homozygous descent. The male and female heterozygotes are then crossed in order to generate homozygous animals. Unless otherwise indicated, the transgenic animals according to the invention comprise stabile changes in nucleotide sequence of the cells of the germ line.

According to another embodiment of the invention, the non-human transgenic cell according to the invention can be used as a nucleus donor cell in the context of a nucleus transfer or nuclear transfer. Nuclear transfer is understood to mean the transfer of the nucleus of a living vertebrate donor cell of an adult or fetal organism into the cytoplasm of an enucleated recipient cell of the same species or of a different species. The transferred nucleus is reprogrammed in order to direct the development of cloned embryos that can then be transferred to carrier females in order to produce fetuses and newborn or utilized for producing cells from the internal cell mass in culture. Different linear cloning techniques can be utilized; of these, those forming the object of the following patent applications can be mentioned non-exhaustively: WO 95 17500, WO 97 07668, WO 97 07669, WO 98 30683, WO 99 01163, WO 99 37143.

According to one preferred embodiment of the invention, genic screening according to the present invention comprises a “knock-in” (K-I). The transgene or the exogenous gene coding for at least one reporter protein according to the invention is screened for by homologous recombination in the genome of an organism. According to one preferred embodiment, the transgene according to the invention is deprived of elements of regulation of genic expression and is placed under the control of the endogenous elements regulating the expression of the gene coding for the specific protein of at least one type of polarization of the immune response and/or one type of effector function of the immune response. Thus, according to one preferred embodiment of the invention, at least one transgene coding for at least one reporter protein according to the invention is introduced into the genome of an animal cell, preferably murine, under the control of elements regulating the expression of the murine γ-interferon gene and/or under the control of elements regulating the expression of the murine interleukin-4 (IL-4) gene, without invalidating expression of these to endogenous genes.

The transgene comprises at least one gene, called the reporter gene, that codes for the reporter protein according to the invention. The reporter gene comprises either the entirety of sequences containing the information for the regulated production of the corresponding RNA (transcription) or the corresponding polypeptide chain (transcription-translation). The reporter gene can be a wild type gene having a natural polymorphism or for a genetically manipulated DNA sequence, for example, having deletions, substitutions or insertions into the coding or non-coding regions. Preferably, the reporter gene or genes are deprived of sequences necessary for directing and controlling their expression in one or several appropriate cell type(s); in fact, they are placed after homologous recombination under the control of the endogenous animal sequences regulating the expression of the target endogenous animal gene that preferably remains active following the homologous recombination event and the integration of the reporter gene.

Alternatively, the transgene according to the invention can contain appropriate regulatory sequences for directing and controlling the expression of said reporter protein or proteins in the cell. In this case, the transgene is integrated randomly into the genome or it is present in episomal form in the cell. In the case represented in the figure, the appropriate regulatory sequences are sequences that can be induced by one or a plurality of proteins of a specific type or of an immune response polarization type or of an immune response effector type, or by one or a plurality of proteins of the specific metabolic pathway of an immune response polarization type or of an immune response effector type.

Regulatory elements of gene expression are understood to mean all of the DNA sequences implicated in the regulation of genic expression; e.g., essentially the sequences regulating transcription, splicing, translation. Of the DNA sequences regulating transcription, the minimal portion sequence, the sequences upstream (for example, the SP1 box, IRE for “interferon responsive element”, etc.), the activator enhancer sequences (“enhancers”), possibly the inhibitor sequences (“silencers”), the insulator sequences (“insulator”), the splicing sequences can be mentioned.

These sequences regulating expression are operationally linked to the reporter gene(s). A nucleic sequence is “linked operationally” when it is placed in a functional relationship with another nucleic acid sequence. For example, a promoter or an activator (“enhancer”) is linked operationally to a coding sequence, if it affects transcription of said coding sequence. Concerning the “operationally linked” transcription regulatory sequences means that the linked DNA sequences are contiguous and when it is a question of linking two regions coding for proteins, contiguous and in the reading phase.

The transgenic cell and/or transgenic non-human animal according to the invention is obtained by introducing at least one transgene coding for a reporter protein into a cell, a zygote or an early embryo of a non-human animal. The introduction of the different transgenes into the cell according to the invention can also be done simultaneously or offset in time. When the cell contains several transgenes, it can be obtained directly by simultaneous introduction into said cell of the DNA fragments necessary for homologous recombination by using methods favoring co-transformation of multiple DNA molecules. These cells are then selected for the expected multiple recombination events by utilizing an appropriate selection system. Alternatively, the multi-transgenic cell can be obtained by producing the homologous recombination events separately and offset in time. Accordingly, the cell, after introduction of a first homologous recombination vector, is selected for the first homologous recombination event by using an appropriate selection method; this newly transgenic cell is then transformed using a second homologous recombination vector, then selected for the second homologous recombination event using an identical or different method. Optionally, this double transgenic cell may then be transformed using a third homologous recombination vector, then selected for the third homologous recombination event using an identical or different selection system, and so on. Alternatively, the double, triple or multi-transgenic cell according to the invention can be obtained by successive breeding of transgenic animals. For example, a double transgenic cell can be obtained by crossing two homozygous single transgenic animals; it can be obtained by then crossing and then selection of two heterozygous simple transgenic animals, or by crossing and selection of a simple transgenic homozygous animal and a simple transgenic heterozygous animal.

According to another embodiment of the invention, the cell is characterized in that said transgene coding for at least one reporter protein is randomly integrated into said cell without said integration of the transgene invalidating the expression of one or a plurality of genes coding for the proteins involved in polarization of the immune response and/or an effector function of the immune response, nor affecting the biological network of the cell or the animal. In this case, the transgene is preferably integrated into a non-coding region of the genome in dependence on elements of response to proteins involved in polarization of the immune response or in effector functions.

According to another embodiment of the invention, the cell is characterized in that said transgene coding for at least one reporter protein is present in episomal form in said cell. It is within the reach of the specialist in the field to define the nature and characteristics of the expression vector used to make possible maintenance and expression in the episomal form of the transgene in the cell of the invention.

In the context of the present invention, a reporter gene is understood to mean a gene that enables cells comprising this gene to be specifically detected after expression of same, that is, to be distinguished from other cells that do not contain this marker gene. Said reporter gene according to the invention codes for a reporter protein chosen preferably from the group comprised of the auto-fluorescent proteins such as green fluorescent protein (GFP), enhanced green fluorescence protein (EGFP), yellow fluorescence protein (YFP), blue fluorescence protein (BFP), red fluorescence protein (RFP), as well as the variants of these fluorescence proteins obtained by mutagenesis in order to produce fluorescence of a different color. Said reporter gene codes also for any enzyme detectable by fluorescence, phosphorescence, or visibly using a histochemical method on living cells or any other cellular analytical method or by microscopy. β-galactosidase (β-GAL), β-glucoronidase (β-GUS), alkaline phosphatase, especially placental alkaline phosphatase (PLAP), alcohol dehydrogenase, especially Drosophila alcohol dehydrogenase (ADH), luciferase, especially firefly luciferase, choramphenicol acetyl transferase (CAT), growth hormone can be mentioned but not exhaustively.

The present invention relates also to the non-human transgenic animal comprising at least one cell according to the invention. Transgenic animal is understood to mean a non-human animal, preferably a mammal chosen from the group comprising rodents and especially the mouse, the rat, the hamster, the guinea pig. The mouse is particularly valuable because its immune system has been extensively studied. Alternatively, the transgenic animal is chosen from among the breeding animals and especially porcine, ovine, caprine, bovine, equine, especially the horse, the lagomorphs, especially the rabbit. The transgenic animal according to the invention can also be chosen from among the primates, especially the simians such as the macaque, the chimpanzee, the baboon.

Considering the genetic polymorphisms present in the population, it can be interesting for analyzing or obtaining a characteristic physiological or behavioral response that the transgenic animals according to the invention, an especially the transgenic mouse according to the invention present different genetic bases. Accordingly, the mouse according to the invention can be selected in consanguinous murine lines (inbred) 129Sv, 129O1a, C57B16, BalB/C, DBA/2 but also in non-consanguinous (outbred) lines or hybrid lines.

The transgenic animal according to the invention comprises at least one cell whose genome comprises at least [a] transgene according to the invention, present either as an extra-chromosomal element or stabily integrated in the chromosomal DNA. Preferably, the entirety of the animal's cells and especially its germ line cells are transgenic.

According to a preferred embodiment of the invention, the invention relates to a non-human transgenic animal characterized in that it comprises at least one cell expressing (i) a first transgene coding for a first reporter protein, said first transgene being integrated by homologous recombination (knock-in) at the level of an endogenous gene coding for a Th1 specific immune response polarization protein without invalidating the expression of said endogenous gene; and (ii) a second transgene coding for a second reporter protein, distinct from said first reporter protein, said second transgene being integrated by homologous recombination (knock-in) at the level of an endogenous gene coding for a specific type Th2 immune response polarization protein, without invalidating the expression of said endogenous gene, the expression of said second transgene being correlated with the expression of said endogenous animal gene. More preferably still, this non-human transgenic is characterized in that the specific type Th1 immune response polarization protein is γ-IFN, the specific type Th2 immune response polarization protein is interleukin-4 (IL-4), the said first transgene codes for the GFP reporter protein and the second transgene codes for the RFP reporter protein.

Also one of the objects of the invention is to provide an in vitro process for characterizing the type of immune response polarization induced by an immunogen, characterized in that it comprises the steps (a) placing said immunogen in contact with a cell according to the invention; (b) determination if expression or expressions of the transgene(s) coding for at least one reporter protein, whose expression is associated with one type of immune response polarization, is produced (respectively, are produced); (c) optionally, quantitative evaluation of the expression of the reporter protein(s), and finally (d) qualitative, optionally quantitative, characterization of the type(s) of immune response polarization.

The invention also relates to the in vivo method for characterizing the type of immune response induced by an immunogen, characterized in that it comprises the steps of (a) placing said immunogen in contact with an animal according to the invention; (b) determination if expression or expressions of the transgene(s) coding for at least one reporter protein, whose expression is associated with one type of immune response, is produced (respectively, are produced) in at least one cell of said animal; (c) optionally, quantitative evaluation of the expression or of the reporter protein(s) and, finally, (d) qualitative, optionally quantitative, characterization of the type(s) of immune response polarization.

Also one of the objects of the present invention is to provide a method for obtaining non-human animal cells specific to a type of immune response polarization and/or an immune response effector function, characterized in that it comprises the steps of (a) placing an immunogen or a pathogen inducing an immune response and/or an immune response effector function in contact with a cell according to the invention; (b) determining if an expression of the transgene coding for at least one reporter protein, the expression of which is associated with said type of polarization immune response and/or said type of immune response effector function, is produced; and finally (c) identifying the cells expressing said reporter protein.

Also one of the objects of the invention is to provide a method for obtaining non-human animal cells specific to a type of immune response and/or an immune response effector function, characterized in that it comprises the steps of (a) placing an immunogen or a pathogen inducing an immune response and/or an immune response effector function in contact with an animal according to the invention; (b) determining if an expression of the transgene coding for at least one reporter protein associated with said type of immune response and/or with an immune response effector function is produced in at least one cell of said animal; (c) isolating in whole or in part cells of the animal; and, finally, (d) identifying among the cells of said animal the cells expressing said reporter protein. Preferably, the cells are identified and/or characterized by using a cell sorter (flow cytometry) but any other manual or automated means for sorting can be used.

According to one preferred embodiment, the specific immunogen of one type of immune response has been characterized by a process according to the invention as hereinbefore described. Immunogen in the context of the present invention is understood to be a compound capable of triggering an immune response. Of the immunogens, those antigens can be mentioned, which react with the receptors of T and B cells, or with preformed antibodies, or with any other receptor type expressed on the cells involved in the initiation and development of an innate or specific immune response. Of these immunogens, those classically used by the specialist in the field should be mentioned, the allergens, the mitogens, the pathogens, or one of their constituents, of viral, bacterial, parasitic, fungal, mycoplasmic origin; the vaccines and vaccinal components; the excipients, drugs, chemical agents or compounds. Placing a specific immunogen in contact with a cell or animal according to the invention can be accomplished in various ways, for example, by classical infection by a pathogenic microorganism or by biological vector (mosquito, tick, bacteria, virus and parasites or recombinant commensal agent, naked DNA, etc.), by inhalation, by aerosol, by food. Experimentally, the immunogen can be introduced in the animal by systemic route, in particular intravenously, by intramuscular, intradermal route, skin contact or by oral route.

A further object of the invention is to provide a process for screening compounds modulating at least one type of immune response polarization and/or at least one immune response effector function, characterized in that it comprises the steps of (a) placing a cell and/or an animal according to the invention in contact with an immunogen responsible for triggering an immune response, preferably specific of a polarization type, and/or an immune effector function and simultaneously or offset in time, with said compound; (b) placing a cell and/or an animal according to the invention in contact with said immunogen in step (a); (c) qualitative, optionally quantitative, determination of the expression of at least one transgene coding for a reporter protein, whose expression is correlated with one specific type of polarization and/or one effector function and then comparison of said expression(s) triggered in (a) and (b); then (d) identification of the compound that selectively modulates the immune response and/or effector function. According to a preferred embodiment of the invention, said polarization of the immune response is of the Th1 type. According to another preferred embodiment, the said polarization of the immune response is of the Th2 type. According to a preferred embodiment of the invention, the said effector function can be, non-exhaustively, a CTL activity, another cytotoxic function, a phagocytic function, a humoral activity, an immunosuppressive function.

Finally, the invention relates also to the utilization of a composition comprising a compound modulating the immune response, preferably specific to a polarization type and/or an effector function type and a pharmaceutically vehicle acceptable as a medicine for preventive and/or curative treatment of a human being or an animal requiring such treatment, characterized in that the capability of said compound to selectively inhibit or activate the immune response specific to one polarization type and/or one effector function is determined by (a) bringing a cell and/or an animal according to the invention in contact with the immunogen responsible for triggering an immune response, preferably specific to one polarization type and/or effector function and, simultaneously or offset in time, with said compound; (b) placing a cell and/or an animal according to the invention in contact with an immunogen responsible for triggering an immune response, preferably specific to one polarization type and/or effector function; (c) qualitative, optionally quantitative, determination of the expression of at least one transgene coding for a reporter protein, whose expression is correlated with a specific type of polarization and/or effector function and then comparison of said expressions triggered in (a) and (b); then (d) identification of the compound that selectively modulates the immune response specific to one polarization type and/or one effector function. Preferably, said immune response is of the Th1 type. The composition according to the invention can be used in a preventive or curative treatment of a certain number of pathologies for which dysfunction of an immune response polarization type, especially Th1, has been observed. Of these pathologies, allograft rejection, multiple sclerosis, chronic inflammatory intestinal and/or rectocolon disease (CID), Crohn's disease, sarcoiditis, peptic ulcer induced by Helicobacter pylori, rheumatoid arthritis, reactive arthritis, especially Lymes disease.

According to a further preferred embodiment, said immune responses is type Th2 and the said treatment is intended for treatment of the selected pathology in the group comprised tolerance of allografts, passage of HIV infection or confirmed AIDS, Omenn syndrome, atopic disease (IgE mediated allergy), such as immediate hypersensitivity and/or inflammatory reactions, especially systemic anaphylaxis, cutaneous anaphylaxis, especially psoriasis, the chronic inflammatory diseases of the intestine and/or colo-rectum (CID), parasitic disease, in which an IgE response is known to be protective, especially the helminthic parasitic diseases (infections by Schistosoma mansoni and Nippostrongylus filaria), progressive systemic sclerosis, chronic peridontitis, tumoral progression. Pharmaceutically acceptable vehicle is understood to mean any type of vehicle usually used in the preparation of pharmaceutical and vaccinal compositions, e.g., a diluent, synthetic or biological vector, a suspension agent such as isotonic or buffered saline solution. Preferably, these compounds are administered systemically, in particular by intravenous, intramuscular, intradermal or by oral route. Their optimal forms of administration, dosages and galenic forms can be determined according to the criteria generally taken into consideration in the establishment of a treatment adapted to a patient such as, for example, age or body weight of the patient, the severity of his general condition, tolerance to the treatment and known secondary effects, etc. When the agent is a polypeptide, an antagonist, a ligand, a polynucleotide, for example an antisense composition, a vector, for example an antisense vector, it can be introduced into the host tissues or the cells in a certain number of ways, including viral infection, micro-injection or vesicle fusion. Jet injection can also be used for intramuscular administration.

Finally, the invention relates to the use of a cell or an animal according to the invention for the purpose of experimental research for the analysis and study of molecular, biological, biochemical, physiological and/or pathophysiological mechanisms of at least one type of immune response polarization and/or one type of immune response effector function. As a function of the type of research that is to be developed, either an entire animal or cells derived from said animal are used. The cells can be either freshly isolated from the animal or can be immortalized in culture, or multiple passages, or by transforming the cells by using viruses such as the SV40 virus or the Epstein-Barr virus.

Other characteristics and advantages of the invention will become apparent when reading the description that follows together with the examples given hereinafter. In these examples, reference will be made to the following figures:

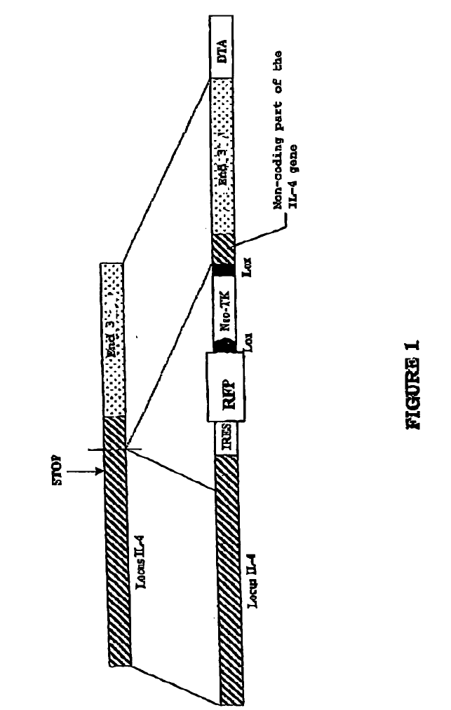

FIG. 1: Diagrammatic representation of the transgene coding for the RFP auto-fluorescent protein for knock-in at the non-coding 3′ extremity for the IL-4 gene.

To: Diagrammatic representation of the 3′ end of the IL-4 gene. The transcribed genic region is shown in broken lines. The region downstream of the IL-4 gene that contains the regulatory sequences is shown in dotted lines. The translation stop site is indicated by STOP.

Bottom: diagrammatic representation of the transgene.

IRES:internal site of entry of the ribosomes of the encephalomyocarditis virus.

RFP: red fluorescence protein

Lox: site of action of Cre recombinase

Neo: Neomycin resistance gene in dependence on the eukaryotic promoter

TK: Thymidine kinase gene

DTA: Diphtheria A toxin

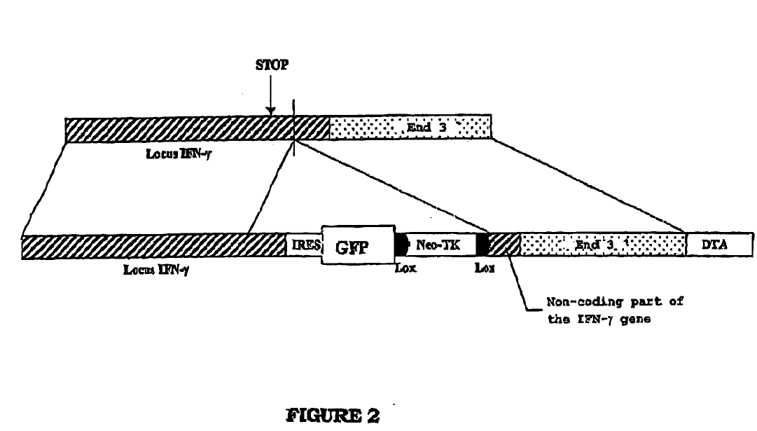

FIG. 2: Diagrammatic representation of the transgene coding for GFP auto-fluorescence protein for knock-in at the non-coding 3′ end of the γ-IFN gene.

Top: diagrammatic representation of the 3′ end of the γ-IFN gene. The genic transcribed region is represented in broken lines. The region downstream of the γ-IL [sic] gene containing the regulatory sequences is represented in dotted lines. The translation stop site is indicated by STOP.

Bottom: Diagrammatic representation of the transgene.

IRES: Internal site of entry of the encephalomyocarditis ribosomes.

RFP: Red fluorescence protein

Lox: Site of action of Cre recombinase

Neo: Neomycin resistance gene in dependence on the eukaryotic promoter

TK: Thymidine kinase gene

DTA: Diphtheria A toxin

### EXAMPLES

### Example 1

### Material and Methods

1.1 Target Endogenous Genes

1.1.1 Interleukin 4

Interleukin 4 (IL-4) is a protein largely secreted, for example, by the activated mastocytes (Bradding et al., 1992) and T-cells (Mossmann et al., 1986). IL-4 plays an important role in differentiation of the naive murine CD4+ T-cells into the effector cells of the Th2 response (Moosmann et al., 1986; Swain et al., 1990; Gross et al., 1993; Kopf et al., 1993).

The murine gene coding for interleukin-4 is located on chromosome 7 of the mouse genome, where it is organized into 4 exons and 4 introns with promoter sequences identified at the 5′ end between positions −760 and −1 (Otsuka et al., 1987). The mechanism of the expression of interleukin-4 involves a certain number of transcription factors that includes GATA-3, C-MAF, NFAT, NIP-45 and JUN B (Li-Weber et al., 1997).

A certain number of mouse models deficient in IL-4 have been generated (Kuhn et al., 1991; Kopf et al., 1993; Noben-Trauth et al., 1996; Metwali et al., 1996; Hu-Li J. et al., 2001). It appears that the phenotype of adult mice, homozygote zero (knock-out) for the interleukin-4 gene, has a reduced IgG1 and IgE level with normal development of the B and T cells and altered Th2 responses. The heterozygotes having, relative to same, responses having an intermediate phenotype.

A cDNA fragment of 585 base pairs coding for mouse IL-4 has been isolated using a cDNA bank obtained using T-helper cells. This cDNA fragment codes for a protein comprised of 140 amino acids (Lee et al., 1986) that, after glycosylation, has a molecular weight of 20 kDa (Sideras et al., 1987).

1.1.2 γ-Interferon

A cDNA of 1292 bp coding for the mouse γ-IFN has been identified by homology with human γ-IFN by screening of a mouse λ phage library (Gray et al., 1983). It codes for a 15 kDa protein secreted exclusively in the form of a non-covalent homodimer. The T-cells (majority of the CD8+, CD4+, Th0 and Th1) and the macrophages are the principal source of γ-IFN. γ-IFN is frequently designated as an inflammatory cytokine because of its property of activating macrophages and increasing the expression of MHC Class II surface molecules and inducing the release of pro-inflammatory cytokines (Farrar et al., 1993). The gene coding for mouse γ-IFN has been located to chromosome 10. It is organized into 4 exons and 3 introns preceded by a 4 kb promoter region at 5′ (Gray et al., 1983). A novel transcription factor called T-bet has recently been identified as being responsible for the induction of the Th1 response by the mediation of γ-IFN production (Szabo et al., 2000). Homozygous mice deficient in γ-IFN develop normally and are healthy in the absence of pathogens. They have increased susceptibility to micro-bacterial infection due to altered macrophage functions (Dalton et al., 1993). The heterozygous animals have a more moderated phenotype.

1.2 Reporter Genes

Intracellular marking using specific monoclonal antibodies is a commonly used method for detecting cytokines in a given cell type. However, the current protocols require protracted procedures that comprise an in vitro culture step accompanied by re-stimulation, followed by fixation of the cells and their permeabilization, and involving cell death of the cells studied. In contrast, following a fluorescent marker combined with the production of the targeted cytokine would make possible almost instantaneous identification of the producing cells with a minimum of manipulation and isolation and collection of viable producing cells (by using flow cytometric sorting, for example).

Numerous mutants have been derived from the native form of the green fluorescent protein (GFP) of the medusa, Aequoria victoria, and are commercially available from different suppliers. Th two reporter genes are chosen so as to produce expression of proteins having high fluorescent intensity detectable by flow cytometry simultaneously using two different wavelengths. The mutant given the highest intensity of fluorescence will be associated with IL-4 produced in lesser quantity relative to γ-IFN.

The expression of the auto-fluorescence proteins using transgenes does not appear to induce significant toxicity or an alteration of the immune response. The transgenic mice of the aforementioned type express GFP or a GFP mutant appear to develop normally. The auto-fluorescent proteins according to the invention are largely sequestered to the interior of the cell.

Preferably, the reporter genes chosen are GFP and RFP. In fact, this pair makes possible detection by the majority of commercially available flow cytometers. The expression of the RFP protein is preferably combined with the IL-4 protein and that of GFP with γ-interferon.

1.3 Homologous Recombination Vector

A vector containing the gene coding for the desired fluorescent protein will be transfected in embryonic mouse cells chosen on an appropriate genetic basis in order to carry out homologous recombination of the vector in the locus of the target cytokine.

It has been shown that the internal ribosome entry site (IRES for Internal ribosome entry site) of the encephalomyocarditis virus (EMCV for encephalomyocarditis virus) enabling simultaneous translation using one single mRNA transcript, of two reporter genes placed under the control of one same promoter (Mountford et al., 1995; Zhu et al., 1999; Liu et al., 2000). This module has been recently used for obtaining in activated mouse T cells a linear relation between IL-4 expression and that of the GFP gene introduced by retroviral infection (Costa et al., 2000). In the system proposed by the inventors, a vector containing the reporter gene under the influence of an IRES sequence will be introduced by knock-in following of or at the locus of the gene of the screened cytokine. In order to keep to a minimum any perturbation of the natural mechanisms of secretion of the targeted cytokines, the IRES reporter gene constructions must be introduced into a portion of the gene that does not contain known regulatory sequences. Positive selection markers (such as the gene for neomycin resistance) and negative selection markers (such as DTA or tk) are also introduced into the vector in order to select embryonic cells having completed homologous recombination. Lox sequences are added in order to make possible excision of the selection markers by the action of Cre; this is done in order to obtain a transgenic animal in which selection genes are absent.

1.4 Transgenic Mice

Transgenic mice expressing one or the other of the reporter genes (one associated with γ-IFN and the other associated with IL-4) are produced independently. The homozygotes for each type of transgenic are then crossed and the descendants are tested in order to select the animals expressing the two transgenics.

The genetic base of the transgenic animals can also be changed by successive breeding with animals of genetic base other than the one initially used.

### REFERENCES

Bradding et al. (1992) J. Exp. Med. 176 : 1381.

Capecchi (1989) Science 244 : 1288.

Costa et al. (2000) J. Immunol. 164 : 3581.

Dalton et al. (1993) Science 259 : 1739.

Del Prete et al. (1991) J. Clin. Invest. 88 :346

Farrar (1993) Annu. Rev. Immunol. 11 : 571.

Firestein et al. (1989) J. Imm. 143 : 518.

Gordon et al: (1989) Transgenic Animals, Intl. Rev. Cytol. 115 : 171.

Cray (1983) Proc. Natl. Acad. Sci. USA 80 : 5842.

Gross et Paul (1993) J. Immunol. 150 : 2112.

Groux et al. (1997) Nature 339 : 152.

Hu-Li J. et al. (2001) Immunity 14 : 1-11.

Keon et al. (1990) Methods and Enzymology 185 : 527

Kilby et al. (1993) TIG 9 : 413.

Kopf et al. (1993) Nature 362 : 245.

Kuhn et Muller (1991) Science 254 : 707.

Lavitrano et al. (1989) Cell 57 :717.

Lee et al. (1986) Proc. Natl. Acad. Sci. USA 83 : 2061.

Li-weber et al. (1997) Immunobiology 198 :170.

Liu et al. (2000) Anal. Biochem. 280 : 20.

Lo (1983) Mol. Cell. Biol. 3 : 1803

Mansour et al. (1989) Ann. Res. Genet. 23 : 199.

Metwali et al. (1996) J. Immunol. 157 : 4546.

Mosmann et al. (1986) J. Immunol. 136 : 2348.

Mosmann et al. (1989) Ann. Rev. Immunol. 7 : 145.

Mosmann (1996) Immunol. Today 17 : 138.

Mountford (1995) Trends In Genetics 11 : 179.

Mzoz et al. (1993) Human Gene Ther. 4 : 589.

Neddleman et al. (1970) J. Mol. Biol. 48 : 443

Noben-Trauth et al. (1996) Transgenic Res. 5 : 487.

Otsuka et al. (1987) Nucleic Acids Rea. 15 : 333.

Ouyang et al. (2000) Immunity 0.12:27.

Pearson et al. (1988) Proc. Natl. Acad. Sci. USA 85: 2444.

Ranganath et al. (1998) J. Immunol. 161 : 3822.

Robinson et al. (1993) J. 1 mm. 143 :518.

Romagnani (1999) Inflamm. Bowel Dis. 5 : 285.

Sambrook et al. (1989) Molecular cloning: a laboratory manual second edition—Cold Spring Harbor Laboratory Press. Cold Spring Harbor, N.Y. USA

Sauer (1994) Current opinion in Biotechnology 5 : 521.

Sauer B. (1998) Methods 14 : 381-392.

Sideras et al. (1987) Adv. Up. Med. Biol. 213 :227.

Smith et Waterman (1981) Ad. App. Math. 2 : 482

Sorscher et al. (1994) Gene Therapy 1 : 223-238.

Swain et al. (1990) J. Immunol. 145 : 3796.

Szabo et al. (2000) Cell 100 : 655.

Thompson et al. (1989) Cell 56 : 313.

Van der putten et al. (1985) Proc. Natl. Acad. Sci. USA 82 : 6148.

Wei et al. (1994) Human Gene Ther. 5 : 969-978.

Wiernenga et al. (1990) J. 1 mm. 144 : 4651.

Yamamura et al. (1991) Science 254 : 77.

Zheng (1997) Cell 89 : 587.

Zhu et Grace (1999) Cytometry 37 : 51.

1. Non-human transgenic animal cell expressing at least one transgene integrated into the genome of the said cell in a stable manner, and coding for at least one reporter protein, characterised in that:
the expression of the said reporter protein is correlated to the expression of at least one protein produced naturally by the said cell and specific to a type of polarisation of the immune response and/or an effector function of the immune response,
the expression of the said transgene is controlled by the regulation elements of the coding endogenic animal gene for the said naturally produced protein and specific to a type of polarisation of the immune response and/or an effector function of the immune response.

2. Cell according to claim 1, characterised in that the said transgene, or the expression of the said transgene, does not affect the biological network of the said cell.

3. Cell according to claims 1 and 2, characterised in that there is a distinct reporter protein for each type of polarisation of the immune response.

4. Cell according to claims 1 and 2, characterised in that there is a distinct reporter protein for each type of effector function of the immune response.

5. Cell according to claims 1 to 4, characterised in that the type of polarisation of the immune response is chosen from among the T helper type (auxiliary T), T repressor, T cytotoxic, the NK type, K type and the humoral type.

6. Cell according to claim 5, characterised in that the T helper type will be chosen from among types Th0, Th1, Th2, Th3 and Tr1.

7. Cell according to claim 6, characterised in that the T helper type will be chosen from among types Th1 and Th2.

8. Cell according to claim 7, characterised in that the said cell expresses two coding transgenes each coding for a distinct reporter protein, the expression of each reporter protein being specific to a polarisation type Th1 or Th2 of the immune response.

9. Cell according to claims 1, 2 and 4, characterised in that the type of effector function of the immune response is chosen from among CTL activities or functions, phagocyte activities or functions, cytotoxic activities or functions, immunosuppressive activities or functions, antigen presentation activities or functions, and cellular activation activities or functions.

10. Cell according to claims 1 to 9, characterised in that the said transgene is integrated by homologous recombination (“Knock-in”) at the said coding endogenic gene for the said protein specific to a type of polarisation of the immune response and/or an effector function of the immune response, without inhibiting the expression of the said endogenic animal gene.

11. Cell according to claim 10, characterised in that the said transgene is integrated on the upstream side, or on the downstream side, or in the middle of the encoding gene for the said protein specific to a type of polarisation of the immune response and/or an effector function of the immune response.

12. Cell according to claim 11, characterised in that the said transgene comprises a sequence to retrieve the translation, the said sequence being located between the coding sequence of the said reporter protein and the coding sequence of the said protein specific to a type of polarisation of the immune response and/or an effector function of the immune response.

13. Cell according to one of claims 1 to 9, characterised in that the said transgene is integrated at random without the said integration having any effect on the biological network of the animal and without inhibiting the expression of the said coding genes for proteins specific to a type of polarisation of the immune response, or a type of effector function.

14. Non-human transgenic animal cell, characterised in that it expresses:
(a) a first coding transgene for a first reporter protein, the said first transgene being integrated by homologous recombination (“Knock-in”) in a coding endogenic animal gene for a specific protein with type Th1 polarisation of the immune response without inhibiting the expression of the said endogenic gene, the expression of the said first transgene being correlated with the expression of the said endogenic animal gene; and/or
(b) a second coding transgene for a second reporter protein, distinct from the said first reporter protein, the said second transgene being integrated by homologous recombination (“Knock-In”) at a coding endogenic gene for a specific protein with type TH2 polarisation of the immune response, without inhibiting the expression of the said endogenic gene, the expression of the said second transgene being correlated with the expression of the said endogenic animal gene.

15. Cell according to one of claims 1 to 14, characterised in that the said reporter protein is selected from among the group consisting of auto-fluorescent proteins and enzymes that can be detected by a histochemical method.

16. Cell according to claim 15, characterised in that the said auto-fluorescent protein is chosen from among the group composed of green fluorescent protein (GFP), enhanced green fluorescent protein (EGFP) red fluorescent protein (RFP), blue fluorescent protein (BFP), yellow fluorescent protein (YFP) and fluorescent variants of these proteins.

17. Cell according to claim 15, characterised in that the said enzyme is chosen from among the group composed of β-galactosidase, β-glucoronidase, alkaline phosphatase, alcoholic dehydrogenase, luciferase, chloramphenicol-acetyl-transferase, and growth hormone.

18. Cell according to one of claims 1 to 17, characterised in that the said specific protein with type Th1 polarisation is chosen from the group composed of interleukin 2 (IL-2), interleukin 12 (IL-12), interleukin 18 (IL-18), interferon-γ, TNF-β, TNF-α T-bet, STAT-4, the β chain of the interferon γ receiver (IFN-γ), receiver chains with IL-12 and IL-18, RANTES, MIP-1α, MIP-1β, CD26, the β2 chain of the interleukin 12 receiver (IL-12Rβ₂), CCR5, CCR2, CXCR3.

19. Cell according to claim 18, characterised in that the specific protein for type Th1 polarisation is IFN-γ.

20. Cell according to claim 19, characterised in that the said specific protein for type Th1 polarisation is IFN-γ and the reporter protein is GFP.

21. Cell according to any one of claims 1 to 17, characterised in that the specific protein for type Th2 polarisation is chosen from the group composed of interleukin 3 (IL-3), interleukin 4 (IL-4), interleukin 5 (IL-5), interleukin 10 (IL-10), interleukin 13 (IL-13), GATA-3, STAT-6, c-maf, NFAT, NIP45, CD30, CD26L, ST2L, CCR3, CCR8, CXCR4, CRTH2 and STIF.

22. Cell according to claim 21, characterised in that the specific protein for type Th2 polarisation is interleukin 4 (IL-4).

23. Cell according to claim 22, characterised in that the said specific protein for type Th2 polarisation is IL-4 and the reporter protein is RFP.

24. Cell according to one of claims 1 to 23, chosen from among the group composed of mouse, rat, hamster, guinea pig, rabbit, primate, ovine, caprinae, bovine porcine and horse cells.

25. Mouse cell according to claim 24.

26. Cell according to one of claims 1 to 25, characterised in that the said cell is chosen from among cells in the immune system, neuron cells, embryo stem cells, haematopoietic stem cells, and neuron stem cells.

27. Cell according to claim 26, characterised in that the said cell in the immune system is chosen from among T lymphocytes, NK cells, K cells, B lymphocytes, mastocytes, macrophages, monocytes, neutrophiles, eosinophiles, basophiles, blood platelets, dendritic cell monocytes and Langerhans cells.

28. Cell according to claim 26, characterised in that the said stem cell is substantially differentiated in a cell chosen from among the cells of the immune system according to claim 27 and neuron cells.

29. Non-human transgenic animal comprising at least one cell according to claims 1 to 28.

30. Animal according to claim 29, characterised in that it is chosen from among the group composed of mice, rats, hamsters, guinea pigs, lagomorphs, primates, sheep, goats, cattle, pigs and horses.

31. Animal according to claim 30, characterised in that the animal is a mouse.

32. Non-human transgenic animal according to claim 31, characterised in that it comprises at least one cell expressing:
(a) a first coding transgene for a first reporter protein, the said first transgene being integrated by homologous recombination (“Knock-in”) in a coding endogenic animal gene for a specific protein with type Th1 polarisation of the immune response without inhibiting the expression of the said endogenic gene, the expression of the said first transgene being correlated with the expression of the said endogenic animal gene; and/or
(b) a second coding transgene for a second reporter protein, distinct from the said first reporter protein, the said second transgene being integrated by homologous recombination (“Knock-In”) at a coding endogenic gene for a specific protein with type TH2 polarisation of the immune response, without inhibiting the expression of the said endogenic gene, the expression of the said second transgene being correlated with the expression of the said endogenic gene.

33. Non-human transgenic animal according to claim 32, characterised in that the said specific protein for type Th1 polarisation of the immune response is IFN-γ and the said specific protein for type Th2 polarisation is interleukin 4.

34. Non-human transgenic animal according to claim 33, characterised in that the said first transgene codes for the GFP reporter protein and in that the said second transgene codes for the RFP reporter protein.

35. In vitro process to characterise the type of polarisation of the immune response induced by an immunogen, characterised in that it comprises the following steps:
a) bring the said immunogen into contact with a cell according to any one of claims 1 to 28;
b) determine if there is an occurrence of one or more expressions of coding transgenes for at least one reporter protein for which the expression is associated with a polarisation type of the immune response;
c) optionally, a quantitative evaluation of the expression of the reporter protein(s);
d) a qualitative, and optionally quantitative, characterisation of the polarisation type(s) of the immune response.

36. In vivo process to characterise the type of the immune response induced by an immunogen, characterised in that it comprises the following steps:
a) bring the said immunogen into contact with an animal according to any one of claims 29 to 34;
b) determine if there is an occurrence of one or more expressions of coding transgenes for at least one reporter protein for which the expression is associated with a type of immune response, in at least one cell in the said animal;
c) optionally, a quantitative evaluation of the expression of the reporter protein(s);
d) a qualitative, and optionally quantitative, characterisation of the polarisation type(s) of the immune response.

37. Method of obtaining non-human animal cells specific to a type of polarisation of the immune response and/or an effector function of the immune response, characterised in that it comprises the following steps:
a) bring the said immunogen or a pathogenic agent inducing the development of an immune response and/or an effector function of the immune response, into contact with a cell according to any one of claims 1 to 28;
b) determine if there is an occurrence of the coding transgene for at least one reporter protein, for which the expression is associated with the said type of polarisation of the immune response and/or the said type of effector function of the immune response;
c) identify cells expressing the said reporter protein.

38. Method of obtaining non-human animal cells specific to a type of immune response and/or an effector function of the immune response, characterised in that it comprises the following steps:
a) bring the said immunogen or a pathogenic agent inducing the development of an immune response and/or an effector function of the immune response, into contact with an animal according to any one of claims 29 to 34;
b) determine if there is an occurrence of the coding transgene for at least one reporter protein associated with the said type of immune response and/or an effector function of the immune response;
c) isolate all or some of the animal cells;
d) identify the cells expressing the said reporter protein, among the cells in the said animal.

39. Method according to claims 37 and 38, characterised in that the said immunogen specific to an immune response type is characterised by a method according to claim 35 or 36.

40. Method according to claims 37 to 39, characterised in that the said cells are identified or characterised by flux cytometry.

41. Method of screening compounds modulating at least one type of polarisation of the immune response and/or at least one effector function of the immune response, characterised in that it comprises the following steps:
a) bring a cell according to claims 1 to 28 and/or an animal according to one of claims 29 to 34 into contact with an immunogen responsible for triggering an immune response and/or an effector function, and with the said compound, either at the same time or with a time lag;
b) bring a cell according to claims 1 to 28 and/or an animal according to one of claims 29 to 34 into contact with the said immunogen in step a);
c) a qualitative, and optionally quantitative, characterisation of the expression of at least one coding transgene for a reporter protein for which the expression is correlated to a specific type of polarisation and/or effector function, and then compare the said expressions obtained in a) and b); and then
d) identify the compound that selectively modulates the immune response and/or an effector function.

42. Method according to claim 41, characterised in that the said polarisation of the immune response is of type Th1.

43. Method according to claim 41, characterised in that the said polarisation of the immune response is of type Th2.

44. Use of a cell according to claims 1 to 28 or an animal according to claims 29 to 34 for the analysis and study of molecular, biological, biochemical, physiological and/or physiopathological mechanisms of at least one type of polarisation of the immune response, and/or a type of effector function of the immune response.

45. Transgene comprising all or part of the murine IL-4 gene, characterised in that a coding sequence for an auto-fluorescent protein, a positive selection cassette that may or may not be surrounded by sites specific to the action of recombinases, is inserted in this order into the non-coding end 3′ of the murine IL-4 gene, and is characterised in that a negative selection cassette DTA and/or TK is present at one or more ends of the transgene.

46. Transgene comprising all or part of the murine IFN-γ gene, characterised in that a coding sequence for an auto-fluorescent protein, a selection cassette that may or may not be surrounded by sites specific to the action of recombinases, is inserted in this order into the non-coding end 3′ of the murine IFN-γ gene, and is characterised in that a negative selection cassette DTA and/or TK is present at one or more ends of the transgene.
